(19) United States
(12) Patent Application Publication
(10) Pub. No.: US 2010/0100060 A1
(43) Pub. Date: Apr. 22, 2010

(54) APPLICATOR FOR PHARMACEUTICAL PRODUCT AND METHOD OF USING SAME

(75) Inventor: BARRY TURNER, Oakland, NJ (US)

Correspondence Address: FRANK A. SMITH;Novartis Consumer Health, Inc., 200 Kimball Drive, OTC PATENT DEPARTMENT - 5TH FLOOR, Parsippany, NJ, 07054-0622, US

(73) Assignee: NOVARTIS AG, Basel (CH)

(21) Appl. No.: 12/580,842

(22) Filed: Oct. 16, 2009

Related U.S. Application Data
(60) Provisional application No. 61/196,503, filed on Oct. 17, 2008.

(51) Int. Cl. A61M 35/00

(52) U.S. Cl. 604/290; 604/310

(57) ABSTRACT

A hand-held applicator is defined for applying a viscous product, such as a pharmaceutical product to the skin. The applicator includes a substrate with a flat face defining multiple dosing areas of the product. The product can be dispensed from a tube in a first area for a first recommended dosage and in combination in a first area and a second area for a second recommended dosage. Once the desired dosage is dispensed, the applicator is used to apply to product to an affected area of the skin.

### FIELD OF THE INVENTION

This application relates to the field of applicators for topical products. In particular, it is a hand-held applicator for applying recommended dosages of a pharmaceutical product to skin.

### BACKGROUND

With an increasing number of medications and topical products on the market today, users are looking for more effective and convenient methods for applying such products. In particular, users are looking for cost-effective applicators that make it easy to dispense a desired dosage and then to apply this dosage to a portion of the body. In addition, with pharmaceutical products, it is often important to deter over-dosing and under-dosing. Thus, users are looking for a cost-effective applicator that allows them to easily apply an appropriate dosage of medication.

### BRIEF SUMMARY

The present invention is a hand-held applicator for applying a topical product, such as a pharmaceutical product, in particular a non-steroidal anti-inflammatory drug, to skin. The applicator is in the form of a substantially flat substrate, having a first face and an opposing second face. At least one of the faces defines a dosing area corresponding to at least one recommended dosage of the product. The product is applied within the dosing area, for example, by being squeezed from a tube. A user then rubs the applicator containing the product thereon, onto an affected area of the skin.

The present invention also encompasses the combination kit of a topical product and an applicator for administering at least one recommended dosage of the product. The product is retained in a dispensing package and the applicator is comprised of a substrate having a front face and an opposing rear face, the front face defining an area corresponding to at least one recommended dosage of the pharmaceutical product. In particular, the kit comprises a dispenser containing a topical product and an applicator for administering at least one recommended dosage of the topical product. The applicator comprises a substrate having a substantially flat front face and an opposing substantially flat rear face. The front face defines a dosing area corresponding to at least one recommended dosage of the topical product and the front face is comprised of a material adapted to retain the topical product without causing the topical product to substantially stick to the surface of the front face and adapted to not substantially change a chemical property of the topical product.

The present invention also encompasses an applicator for topically administering to the skin a viscous pharmaceutical product comprising diclofenac or a pharmaceutically acceptable salt thereof. The applicator comprises a substrate having a front face and an opposing rear face, the front face defining a dosing area corresponding to at least one recommended dosage amount of the pharmaceutical product.

The present invention also encompasses a set of applicators combined together, such as in a booklet, with each individual applicator adapted to administer at least one recommended dosage of a product to a user's skin. The set of applicators comprises a plurality of disposable substrates each having a substantially flat front face and a substantially flat opposing rear face, the front face defining at least one recommended dosage for the product.

The present invention also encompasses a method of using the aforementioned applicators. In particular, the method comprises disposing the product in a first area, if the product is to be applied to one portion of the body, and disposing the pharmaceutical product in the first area and a second area, if the pharmaceutical product is to be applied to an alternate portion of the body and placing the disposed product on an affected area of the skin.

The present invention further contemplates a combination kit intended to be used for housing a dispenser containing a topical product (the “primary container”), said kit comprising: a carton for receiving the primary container or containers; an applicator or a plurality of applicators which are integral with the carton, and optionally an information-bearing insert.

The invention also includes the a kit after being filled with the primary container or containers, i.e. the kit comprising: a carton, at least one primary container housed within the carton, an applicator or plurality of applicators of the invention which are integral with the carton, and optionally an information-bearing insert.

### BRIEF DESCRIPTION OF THE DRAWINGS

For the purpose of illustrating the invention, there is shown in the drawings a form which is presently preferred; it being understood, that this invention is not limited to the precise arrangements and instrumentalities shown.

FIG. 1 is a plan view of an applicator of the type contemplated by the invention.

FIG. 2 is a perspective view of an applicator with a topical product being deposited thereon in a first recommended dosage.

FIG. 3 is a perspective view of the applicator of FIG. 2 with the topical product being administered to a portion of the upper body.

FIG. 4 is a perspective view of the applicator of FIG. 1 with a topical product being deposited thereon in a second recommended dosage.

FIG. 5 is a perspective view of the applicator of FIG. 4 with the topical product thereon being administered to a portion of the lower body.

FIG. 6 is a perspective view of a user rubbing the topical product on a portion of the lower body.

FIG. 7 is a perspective view of a set of applicators in a booklet.

FIG. 8 is a plan view of an applicator removably connected to another applicator.

### DETAILED DESCRIPTION

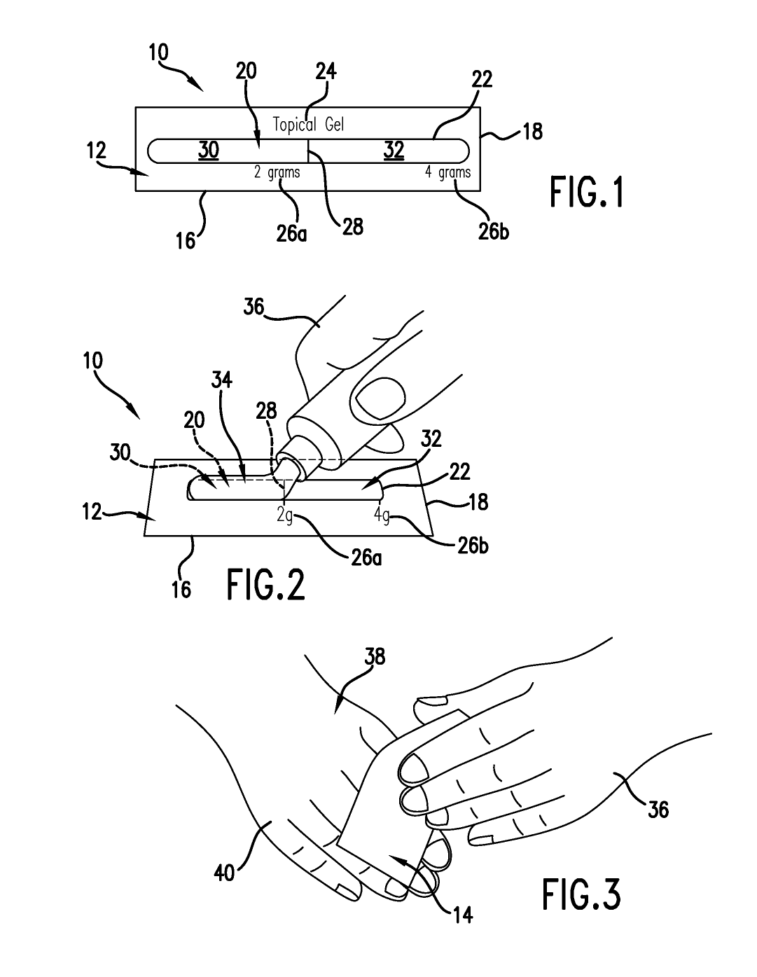

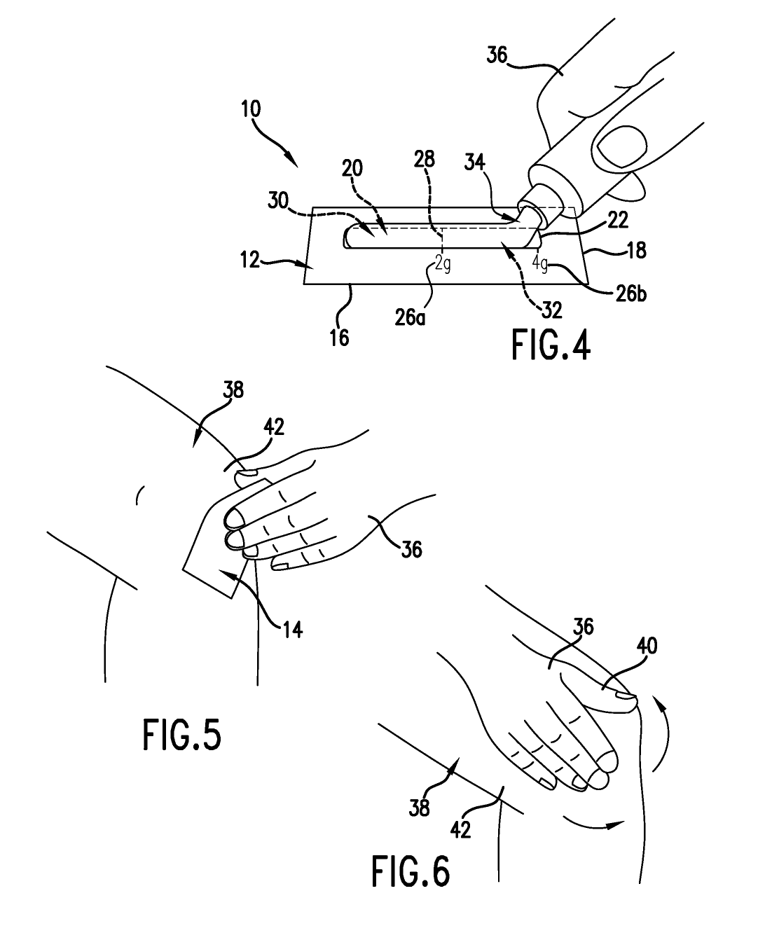

In the figures, where like reference numerals indicate like elements, there is shown multiple embodiments of an applicator for topical products. FIG. 1 shows an applicator 10 for administering a topical product. The topical product is shown in FIGS. 2 and 4 and may be in the form of a pharmaceutical product or solution, in the form of a gel, cream, emulsion or similar viscous composition. The product may be, for example, a diclofenac topical gel commonly sold as the sodium salt form under the trademark VOLTAREN Gel 1% or as the diethylamine salt form under the trademark VOLTAREN EMULGEL 1.16%. Other pharmaceutical or similar products may be used with the applicator. In addition, the applicator is not limited to use with prescription or over-the-counter pharmaceutical products. It may be used with other products such as sunscreen, lotions, etc. The applicator 10 is generally comprised of a substrate in the shape of a rectangle. The substrate has a substantially flat front face 12. The substrate also has a substantially flat rear face 14. As shown in FIGS. 3 and 5, preferably, the applicator 10 is small enough to fit in the hand of a user 36. As shown in FIG. 1, the substrate is defined by a first edge 16 and a second edge 18. The first edge 16 may be about 120 mm long and the second edge 18 may be about 28 mm long. The applicator 10 may have other dimensions. For example, the substrate may be 120 mm by 28 mm when used with a 20 g tube of the product. It will also be understood that the substrate may be other shapes such as a square, oval, circle, rectangle with rounded edges, etc.

At least one of the front face 12 or the rear face 14 defines at least one dosing area 20 corresponding to at least one recommended dose of the product. In the embodiment shown in FIG. 1, the front face 12 has first indicia 22 that defines the dosing area 20. As shown, the dosing area 20 is an elongated area. Dosing areas of any number of other shapes and sizes may be used. The dosing area 20 is substantially in the same plane as the front face 12. The dosing area 20 may alternatively define a cavity or may protrude from the face of the applicator 10. The front face 12 may also contain second indicia 24 for indicating the product to be used with the applicator 10. For example, second indicia 24, may read “VOLTAREN GEL.” The face with the dosing area 20 may also contain third indicia 26a and fourth indicia 26b, explained in more detail below, which indicates dosing portions corresponding to different dosage amounts.

The dosing area 20 has at least one marker 28 that defines two portions 30, 32 corresponding to two recommended dosages of the product. Alternatively, additional markers may be defined to identify more than two recommended dosages. As shown in FIG. 1, the marker 28 creates a left-hand side portion 30, defining a first recommended dosage, and a right-hand-side portion 32, defining a second recommended dosage. Left and right are defined from the perspective of a user looking at the front face 12 with, at least, second indicia 24, third indicia 26a or fourth indicia 26b being positioned upright. As shown, left and right hand portions are adjacent. Alternatively, they may be separated. Third indicia 26a is located at an end of the left-hand side portion 30 (i.e., adjacent the marker 28). Fourth indicia 26b is located at an end of the right-hand side portion 32. Third indicia 26a is a notation of “2 grams” or “2 g” and fourth indicia 26b is a notation of “4 grams” or “4 g.” The indicia 26a, 26b may be changed if the dosing areas change. The marker 28 may be placed approximately half-way along the elongated dosing area 20, which makes the left-hand side portion 30 and the right-hand side portion 32 substantially equal; i.e., each corresponding to 2 grams. However, the marker 28 may be placed in other positions with respect to the area 20, thus, creating larger or smaller dosages.

The left-hand side portion 30 defines a first recommended dosage of 2 grams and the right-hand side portion 32 defines a portion of an additional 2 grams. Thus, as shown in FIG. 2, when a user 36 squeezes product 34 from a tube or the like onto only the left-hand side portion 30 up to the marker 28 (up to the 2 gram line), so that the front face 12 contains about 2 grams of product 34. As shown in. FIG. 4, if the user 36 continues through the right-hand side portion 32 (up to the 4 gram line), the front face 12 will contain about 4 grams of product 34. It will be understood that the elongated area 20 in combination with the opening of the pharmaceutical product tube produces the desired volume (i.e., dosage) of product. The product 34 substantially fills the dosing area 20 in both the length-wise (i.e., along 16) and width-wise (i.e., along 18) directions.

As shown in FIGS. 3 and 5, once the desired dosage of product is placed onto the front face 12, the user 36 rubs the front face 12 on an affected area of the skin 38. In order to facilitate easy rubbing and complete coverage of an area of skin 38, the substrate of the applicator 10 may be made of a pliable material such as paper or a flexible plastic. The paper may be relatively thin and substantially transparent or translucent. The paper may be coated, may be wax paper or a similar material. In other embodiments, the substrate may be made, of a more rigid material such as a relatively rigid plastic (e.g., polypropylene). The applicator 10, preferably, is disposable. However, the applicator 10 may also be cleanable and reusable. Preferably, the substrate is made of a material (or, face 12 is made of a material) that holds the product while not causing too much of the product to absorb or adsorb into the surface. In addition, it is preferred that the substrate be comprised of a material that does not change the chemical composition of or interact with the product. In this regard, front face 12 may be comprised of a substantially non-porous or hydrophobic material. Further, the front face 12 and rear face 14 may comprise the same material or may comprise different materials. In addition, at least a portion of the front face and the rear face may comprise a different material than the remainder of the material of the front face or the rear face. For example, the dosing area 20 may be made of a different material than the remainder of the front face 12.

FIG. 3 shows a user 36 rubbing the product onto a hand 40. FIGS. 5 and 6 show a user 36 rubbing the product onto a knee 42. Where the product 34 is desired to be applied to an affected area above the waist, preferably a relatively smaller dosage is applied. Where the pharmaceutical product 34 is desired to be applied to an affected area below the waist, preferably a relatively larger dosage is applied. (Above the waist is between the user's waist to the head.) Thus, in the example provided 2 grams would be applied to the hand 40 and 4 grams applied to the knee 42.

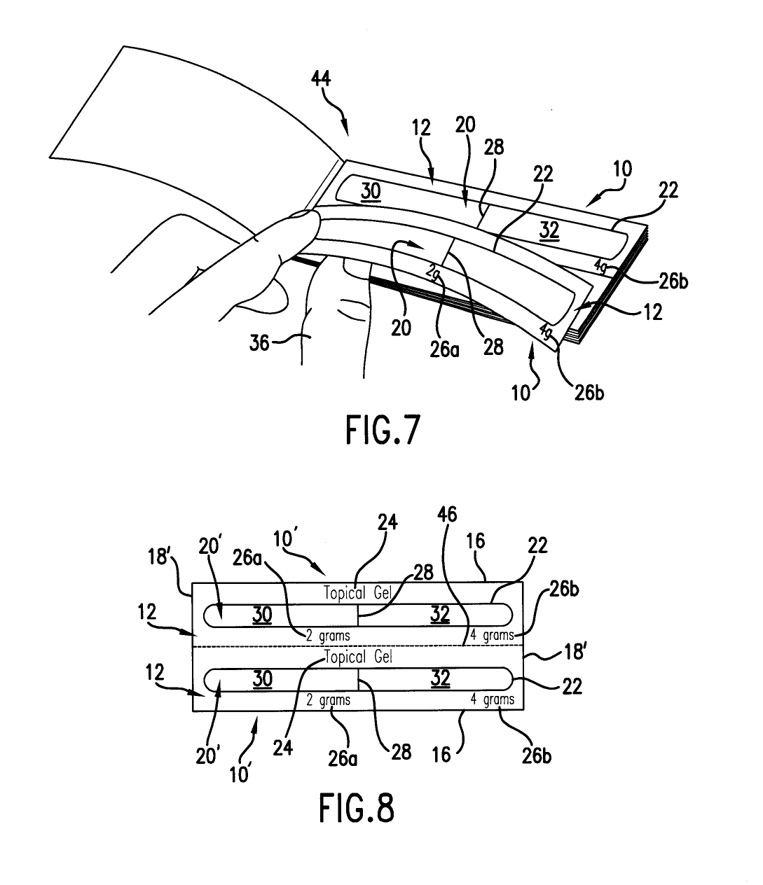

The applicator 10 may be part of a set of applicators, an example of which is shown in FIG. 7. In the set shown in FIG. 7, a plurality of applicators described above are removably disposed in a booklet 44. The applicators 10 may be attached to the booklet 44 adjacent the binding, via a releasable adhesive, or may have a portion that is scored. Other means for releasably securing the applicators 10 to the booklet 44 may be provided. The applicators 10 are disposed such that two applicators 10 make up a “page” of the booklet 44. The applicators 10 are disposed one on top of the other. The applicators 10 may be releasably attached to each other, for example, as being separated by scoring, or the applicators may be separate, as shown in FIG. 7.

FIG. 8 shows another embodiment of the applicator 10′. This embodiment is substantially identical to that shown in FIG. 1 except that the second edge 18′ of the substrate is 17.5 mm. In this embodiment, dosing area 20′ may, therefore, be smaller than dosing area 20 described above. The applicator 10′ may be more suitable for use as a kit with, for example, a 100 g tube of product, whereas applicator 10 may be more suitable for, as an example, a 20 g tube of product.

To use the applicator 10 or 10′, a user places the substrate on a substantially flat surface. The user dispenses a product 34, preferably, on the front face 12 of the substrate in the desired dosage, as described above. Where the applicator is part of a set, as shown in FIG. 7, the user may need to first separate an applicator from the plurality. This may include removing an applicator from the booklet 44. For a 2 gram dosage, a user 36 dispenses the product 34 up the 2 gram line. This is shown in FIG. 2. For a 4 gram dosage, a user 36 dispenses the product 34 through the 2 gram line and up the 4 gram line. This is shown in FIG. 4. The user then takes the applicator 10, 10′ preferably by placing the rear face 14 in the user's hand 40. This is shown in FIGS. 3 and 5. The user may then rub the front face 12, with the product thereon, onto an affected area of the user's skin 38. Alternatively, as shown in FIG. 6, a user may place the front face 12 with the product, on the skin 38, so that the product is transferred to the skin 38. The user then rubs the product in with a hand. Such an affected area of skin may include an area. adjacent parts affected by arthritis. The applicator may then be disposed of or cleaned for reuse

In an illustrative embodiment, the applicator is used to topically administer an effective amount of VOLTAREN Gel 1% to a human for the relief of the pain of osteoarthritis of joints amenable to topical treatment, such as of the knees and those of the hands. For treatment of the lower extremities, the effective amount of gel may be 4 grams (i.e. sufficient to cover entire dosing area 20), applied to the affected area 4 times daily, provided that no more than 16 grams daily is applied to any one affected joint of the lower extremities. For treatment of the upper extremities, said effective amount of the gel may be 2 grams (i.e. sufficient to cover portion 30), applied to the affected area 4 times daily, provided that no more than 8 grams daily is applied to any one affected joint of the upper extremities.

The present invention further contemplates a combination kit intended to be used for housing a dispenser containing a topical product (the “primary container”), said kit comprising: a carton for receiving the primary container or containers; an applicator or a plurality of applicators of the invention which are integral with the carton, and optionally an information-bearing insert.

The invention also includes a kit after being filled with the primary container or containers, the kit comprising: a carton, at least one primary container housed within the carton, an applicator or plurality of applicators of the invention integral with the carton, and optionally an information-bearing insert.

By “integral with the carton” is meant that the applicator or applicators are removably affixed to or otherwise secured to the carton, preferably to an inner surface of the carton. Most preferably, the applicator or applicators are integral with the carton and fully housed within the carton.

The applicator of the invention, as previously described, comprises a substrate having a substantially flat front face defining a dosing area corresponding to at least one recommended dosage of the topical product, and an opposing substantially flat rear face. The dosing area and applicator are preferably defined by an elongated shape. The carton is of a size and shape suitable for containing the one or more primary container(s), and for affixing or securing the applicator(s). The applicator(s) is/are preferably completely contained within the carton and affixed or secured to at least one inner surface. Means for affixing or securing are well-known to the packaging art, e.g., affixing by adhesive or detachable tab or flap, or securing within a pocket or cell formed from at least one surface of the carton, from which the applicator(s) is/are removable by the box user. See, for example, U.S. Pat. Nos. 6,702,108, 6,913,140, 6,923,315, 7,036,615, 5,944,183 WO2000020289, GB 2,330,572, GB 2,352,232, and EP 922,216. The applicator(s) are positioned in a manner so as not to interfere with the insertion of the primary container and optional information-bearing insert into the carton, or with the opening of the carton by the carton user.

The kit is prepared by folding a carton blank to form a carton and including within the carton the applicator(s) of the invention and optional information-bearing insert. In a separate step, the primary container or containers filled with product, is/are inserted into the carton containing the secured applicator(s). The optional information-bearing insert such as a leaflet may be securely housed in the carton when the carton is formed in similar manner to the applicator(s), or alternatively, may be inserted into the carton prior to, during or after filling of the carton with the primary container(s). Typically, the carton is made from a single carton blank made of foldable material, such as cardboard. In one aspect, the applicator is a single, discrete applicator, in which case it is preferably made of fairly rigid material such as polypropylene. In another aspect, the kit comprises a plurality of applicators, such as the booklet shown in FIG. 7, in which case the applicators are preferably made of a readily pliable material such as paper or a flexible plastic.

Advantageously, the kits of the invention reduce the difficulty and expense otherwise encountered by a pharmaceutical company in using pre-formed cartons to package a topical product with accompanying applicator since according to the present invention the applicator is integral with the pre-formed carton as received by the pharmaceutical company from the manufacturer.

Although the applicator has been described and illustrated with respect to the exemplary embodiments thereof, it should be understood by those skilled in the art that the foregoing and various other changes, omissions and additions may be made therein and thereto, without parting from the spirit and scope of the present invention.

What is claimed is:

1. An applicator for administering at least one recommended dosage of a viscous product, the applicator comprising:
a substrate having a front face and an opposing rear face, the front face defining a dosing area corresponding to at least one recommended dosage of the pharmaceutical product.

2. The applicator of claim 1, wherein the substrate is adapted to apply the at least one recommended dosage to skin.

3. The applicator of claim 1, wherein the dosing area corresponding to at least one recommended dosage of the product is defined by an outline of an elongated shape.

4. The applicator of claim 1, wherein the front and rear faces of the substrate are substantially flat.

5. The applicator of claim 1, wherein the substrate comprises a pliable material.

6. The applicator of claim 5, wherein the front face of the substrate is comprised of a substantially hydrophobic material.

7. The applicator of claim 1, wherein the substrate comprises plastic material.

8. The applicator of claim 1, wherein the substrate measures about 28 mm by about 120 mm.

9. The applicator of claim 3, wherein the dosing area further comprises at least one marker separating the dosing area into at least two portions, wherein each portion corresponds to a different recommended dosage of the product.

10. The applicator of claim 9, wherein the marker divides the dosing area in half creating a left-hand-side portion and a right-hand-side portion, wherein the left-hand-side portion corresponds to a first recommended dosage and the right-hand-side portion with the left-hand side portion corresponds to a second recommended dosage.

11. A set of applicators, according to claim 1, each adapted to administer at least one recommended dosage of a viscous product to a user's skin, the set comprising:
a plurality of disposable substrates each having a substantially flat front face and a substantially flat opposing rear face,
wherein the front face defines at least one recommended dosing area for the product.

12. The set of claim 11, wherein each substrate has an outline of an elongated shape that defines an area corresponding to at least one recommended dosage.

13. The set of claim 11, wherein the plurality of substrates are releaseably attached in a booklet.

14. The set of claim 11, wherein each substrate measures about 28 mm by about 120 mm.

15. The set of claim 11 wherein the viscous product comprises diclofenac or a salt thereof, and wherein the at least one recommended dosing area corresponds to a dosage amount of 2 grams or 4 grams.

16. The set of claim 13, wherein each page of the booklet contains two substrates arranged one on top of the other.

17. The set of claim 12, further comprising at least one marker dividing the area substantially in half and defining a first portion and a second portion, wherein the first portion corresponds to a first recommended dosage and the second portion with the first portion, corresponds to a second recommended dosage.

18. The set of claim 17, wherein the first recommended dosage is about two grams and the second recommended dosage is about four grams.

19. An applicator, according to claim 1, for topically administering to the skin a viscous pharmaceutical product comprising diclofenac or a pharmaceutically acceptable salt thereof, the applicator comprising:
a substrate having a front face and an opposing rear face, the front face defining a dosing area corresponding to at least one recommended dosage amount of the pharmaceutical product.

20. The applicator of claim 19, wherein the at least one recommended dosage amount is 2 grams.

21. The applicator of claim 19, wherein the at least one recommended dosage amount is 4 grams.

22. The applicator of claim 19, wherein the dosing area further comprises at least one marker separating the dosing area into at least two portions, wherein each portion corresponds to a dosage amount of 2 grams.

23. A kit comprising:
a dispenser containing a topical product; and
an applicator for administering at least one recommended dosage of the topical product, the applicator comprising:
a substrate having a substantially flat front face and an opposing substantially flat rear face, the front face defining a dosing area corresponding to at least one recommended dosage of the topical product,
wherein the front face is comprised of a material adapted to retain the topical product without causing the topical product to substantially stick to the surface of the front face and adapted to not substantially change a chemical property of the topical product.

24. The kit of claim 23, wherein the material comprising the front face is substantially hydrophobic.

25. The kit of claim 23, wherein the material comprising the front face is wax paper.

26. The kit of claim 23, wherein the substrate, front face and rear face are plastic.

27. The kit of claim 23, wherein the dosing area corresponding to at least one recommended dosage of the product is defined by an outline of an elongated shape.

28. The kit of claim 27, wherein the dosing area further comprises at least one marker separating the dosing area into at least two portions, wherein each portion corresponds to a different recommended dosage of the product.

29. The kit of claim 23, wherein the topical product is a pharmaceutical emulsion.

30. The kit of claim 23, wherein the dispenser is a squeezable tube.

31. A kit for housing a primary container for a topical product and at least one applicator according to claim 1, said kit comprising:
a carton for receiving the primary container,
one or more applicators integral with the carton, and
an optional information-bearing insert.

32. A kit containing a topical product and at least one applicator according to claim 1, comprising:
a carton,
at least one primary container housed within the carton containing the product
one or more applicators integral with the carton, and
an optional information-bearing insert.

33. A method of administering a viscous pharmaceutical product to a user's skin, the method comprising:
providing an applicator comprising a substrate having a front face, an opposing rear face and, on one of the front face or rear face, a first portion corresponding to a first recommended dosage of the pharmaceutical product and a second portion corresponding to a second recommended dosage of the pharmaceutical product;
disposing the pharmaceutical product in the first portion if the pharmaceutical product is to be applied to a lower portion of the body;
disposing the pharmaceutical product in the first portion and the second portion if the pharmaceutical product is to be applied to an upper portion of the body; and
placing the pharmaceutical product on an affected area of the skin.

34. The method of claim 33, further comprising rubbing the face having the pharmaceutical product directly on the affected area of the skin.

35. The method of claim 33, wherein the viscous pharmaceutical product comprises diclofenac or a pharmaceutically acceptable salt thereof, the first recommended dosage is 2 grams and the second recommended dosage is 4 grams.

36. A method for topically administering an effective amount of a viscous pharmaceutical product comprising diclofenac or a pharmaceutically acceptable salt thereof to a human subject for the relief of the pain of osteoarthritis of joints of the lower extremities amenable to topical treatment, comprising applying to the affected area of the lower extremities, 4 times daily, an amount of the product that corresponds to a left-hand portion and right-hand portion of the applicator of claim 10, provided that no more than about 16 g daily is applied to any one affected joint of the lower extremities.

37. A method for topically administering an effective amount of a viscous pharmaceutical product comprising diclofenac or a pharmaceutically acceptable salt thereof to a human subject for the relief of the pain of osteoarthritis of joints of the upper extremities amenable to topical treatment, comprising applying to the affected area of the lower extremities, 4 times daily, an amount of the product that corresponds to a left-hand portion of the applicator of claim 10, provided that no more than about 8 g daily is applied to any one affected joint of the lower extremities.
